(19) United States
(12) Patent Application Publication
(10) Pub. No.: US 2004/0192957 A1
(43) Pub. Date: Sep. 30, 2004

(54) Method of recovering 2-ethylhexyl alcohol from a mixture comprising 2-ethylhexyl acrylate

(76) Inventors: Danita Inez Wickens, Pampa, TX (US); Michael Adam Podraza, Amarillo, TX (US)

Correspondence Address: M. Susan Spiering, c/o Celanese Ltd., IP Legal Dept., IZIP 701, P.O. Box 428, Bishop, TX, 78343, US

(21) Appl. No.: 10/404,860

(22) Filed: Mar. 31, 2003

(51) Int. Cl. C07C 69/52; C07C 29/74

(52) U.S. Cl. 560/205; 568/913

(57) ABSTRACT

A process is disclosed for the production of 2-ethylhexyl acrylate which includes a method of recycling unreacted 2-ethylhexyl alcohol. A liquid side stream draw containing about or greater than 65% alcohol, is taken from the top one third of the light ends recovery tower. By employing a side stream, 2-ethylhexyl alcohol can be recovered and directly recycled to the 2-ethylhexyl acrylate reactor thereby increasing raw material efficiency and avoiding problematic emulsion issues associated with use of other in-process streams.

### FIELD OF THE INVENTION

The present invention is directed to the recovery of unreacted 2-ethylhexyl alcohol in the production of 2-ethylhexyl acrylate.

### BACKGROUND OF THE INVENTION

2-Ethylhexyl acrylate manufacture from acrylic acid and 2-ethylhexyl alcohol is a well-known esterification process. Such process is typically carried out by contacting acrylic acid and 2-ethylhexyl alcohol in a reactor with a solvent such as cyclohexane and a catalyst. Typical catalysts include sulfonic acid type catalysts such as benzene sulfonic acid, methane sulfonic acid, p-toluene sulfonic acid, or sulfuric acid, and phosphoric acid or phosphonic acid catalysts.

A problem in the production of 2-ethylhexyl acrylate is unreacted 2-ethylhexyl alcohol which invariably builds up in the purification train. Resolution of the alcohol build-up has included burning the unreacted alcohol, which results in a direct efficiency loss for the acrylate production process. Alternatively, the unreacted alcohol has been stored in inventory tanks. Prior to use, the unreacted alcohol in the tanks is purified to remove light ends impurities and directed to the reactor for use in the esterification reaction. A direct recycle of the 2-ethylhexyl alcohol with the light ends impurities results in emulsion problems within the reaction system, including the purification process.

An additional problem with the recovery of the unreacted 2-ethylhexyl alcohol is production of 2-ethylhexyl acetate. Acetic acid is a common impurity found in acrylic acid. During the esterification reaction of acrylic acid and 2-ethylhexyl alcohol, the side reaction between alcohol and acetic acid is occurring forming 2-ethylhexyl acetate. 2-Ethylhexyl acetate has approximately the same boiling point as 2-ethylhexyl alcohol making it difficult to separate this impurity from the desired alcohol. Various methods have been employed to recover 2-ethylhexyl alcohol from the acrylic acid, 2-ethylhexyl alcohol reaction. One method involves azeotroping the alcohol with water to remove the desired 2-ethylhexyl alcohol. Regardless of the method employed, removal of close-boiling impurities, especially 2-ethylhexyl acetate, is problematical.

The following references are believed illustrative of the art: U.S. Pat. Nos. 4,280,009; 6,072,076; 5,734,074; 3,882,167; 4,833,267; 3,579,309; 5,659,072; 5,093,520, and EP 795,536.

### SUMMARY OF THE INVENTION

The present invention is directed to a method or process of recovering 2-ethylhexyl alcohol comprising:

a) reacting in a reactor, acrylic acid with 2-ethylhexyl alcohol in the presence of a solvent, an acidic catalyst, and at least one polymerization inhibitor, to form a reaction mixture of primarily 2-ethylhexyl acrylate, acrylic acid, solvent, catalyst, 2-ethylhexyl alcohol and 2-ethylhexyl acetate;

b) directing the reaction mixture to an extraction process to remove at least a portion of the catalyst and at least a portion of the unreacted acrylic acid;

c) directing the mixture of step (b) to a process for removal of solvent and forming a stream comprising 2-ethylhexyl acrylate, 2-ethylhexyl alcohol, 2-ethylhexyl acetate, light ends impurities, and heavy ends impurities;

d) directing the stream of step (c) for removal of light ends impurities forming an overhead stream comprising 2-ethylhexyl acrylate and 2-ethylhexyl alcohol, and a residue stream comprising 2-ethylhexyl acrylate, heavy ends impurities, and 2-ethylhexyl acetate;

e) directing the overhead stream of step (d) to a light ends recovery column wherein the residue is recycled to the light ends removal process of step (d); and,

f) withdrawing a liquid side stream from the top third of the light ends recovery column to obtain 2-ethylhexyl alcohol at a concentration level of about or greater than 65%.

The 2-ethylhexyl alcohol of step (f) may, if desired, be directed to the reactor. It has been found that the light ends impurities comprise 2-ethylhexyl alcohol, 2-ethylhexyl acetate, acetic acid, solvent, heptanol, water, 2-ethylhexyl aldehyde, and acrylic acid. The heavy ends impurities comprise 2-ethylhexyl-3-acryloxy propionate, 2-ethylhexyl-3-(2-ethylhexoxy)-propionate, and polymerization inhibitor(s). The recovered 2-ethylhexyl alcohol from the side stream has been shown to have a concentration of greater than 65%, 75%, and also about or greater than 85%.

The esterification reaction may utilize solvents such as cyclohexane, benzene, heptane, toluene, and 2-ethylhexyl alcohol. The acidic catalyst for the reaction may be selected from the group of benzene sulfonic acid, methane sulfonic acid, sulfonic acid resin, p-toluene sulfonic acid, and sulfuric acid and also phosphonic acid catalysts such as phosphoric acid, and polyphosphoric acid, and the like. It is preferable to employ at least one polymerization inhibitor in the reaction system, which also includes adding at least one inhibitor to the purification system. Exemplary inhibitors include air, air blend gas containing at least 5% oxygen, hydroquinone, mono methyl ether of hydroquinone, phenothiazine (PTZ), methoxy benzene, ethoxybenzene, phenol, hydroxylbenzene, sulfhydrylbenzene, aminobenzene, C₁₋₉ alkylbenzene, p-methoxyphenol (MEHQ), tertiary butyl catechol and di-tertiary catechol, a metal cation of manganese, copper, chromium, cerium, iron, TEMPO, and combinations thereof.

Extraction of the 2-ethylhexyl acrylate employs water or water and at least one other solvent such as sodium hydroxide, potassium hydroxide and ammonia at a normality of about 0.1-3.0.

An alternate embodiment of the present invention involves production of 2-ethylhexyl acrylate in a manner known to those of skill in the art and adding the inventive process disclosed herein for the recovery and reuse of 2-ethylhexyl alcohol. Therefore, a stream comprising as primary components 2-ethylhexyl acrylate and 2-ethylhexyl alcohol is fed to a light ends recovery tower so as to withdraw as a liquid side stream, from the top third of the tower, a stream comprising at least 65% 2-ethylhexyl alcohol, about 10-20% 2-ethylhexyl acetate, about 5-15% 2-ethylhexyl acrylate, and light end impurities other than acrylic acid at a concentration of less than about 2%, and wherein further, the top of the tower operates at a temperature generally about 70-100° C., while the base temperature of the tower operates at about 124-142° C. and the tower is under a pressure of about 30-40 mmHg absolute.

It has been found that the top third of the light ends recovery column is optimum for taking a liquid side stream draw of 2-ethylhexyl alcohol. It is desirable to have the side stream draw above the feed, but below the top tray of the column since this is where 2-ethylhexyl alcohol will be most concentrated. If one tries to remove the alcohol as an overhead stream, light end impurities are present. If one attempts to take a side stream draw too low in the column, acrylate product will be extracted instead of the desired alcohol. This discovery avoids the need to further purify the alcohol prior to reuse in the esterification reaction. If desired, the 2-ethylhexyl alcohol may be directed to the reactor for immediate reuse in the esterification reaction.

### BRIEF DESCRIPTION OF THE DRAWINGS

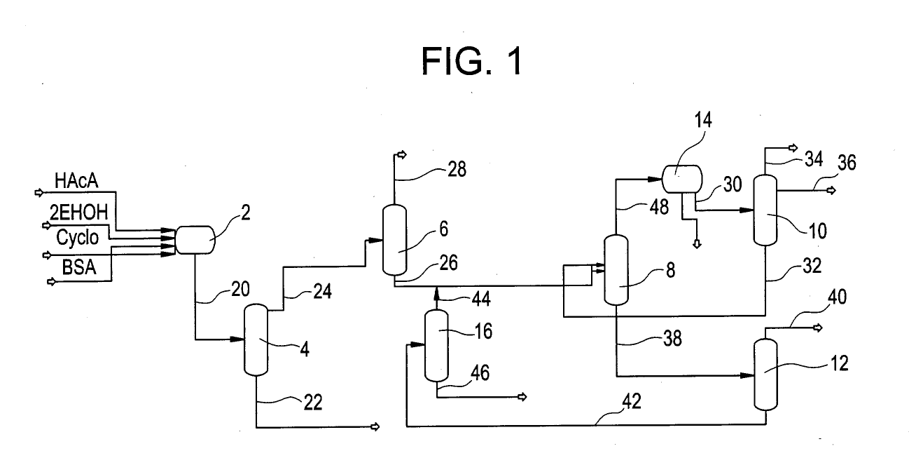

The invention is described in detail below with reference to FIG. 1 which is a schematic diagram illustrating the production of 2-ethylhexyl acrylate and a liquid side stream withdrawal of 2-ethylhexyl alcohol from the light ends recovery tower.

### DETAILED DESCRIPTION OF THE INVENTION

Unless clear from the context, preponderant, primarily, or predominant component and the like refers to a component making up more than about fifty percent (50%) by weight of a mixture;

Unless otherwise specified or clear from the context: percent, ppm and the like refer to parts by weight.

The following are applied herein:

BSA means benzene sulfonic acid.

HAcA means acrylic acid.

Cyclo means cyclohexane.

2-EHOH means 2-ethylhexyl alcohol.

2-EHAc means 2-ethylhexyl acetate.

2-EHAcA means 2-ethylhexyl acrylate.

HAc means acetic acid.

The present invention will be described with reference to FIG. 1. Pressure ranges disclosed are for the operation of the top of the respective towers, and units are in mmHg absolute (abs).

Although a batch reactor was employed, those of skill in the art will recognize that a continuous reactor may also be used for the esterification reaction. Acrylic acid, 2-ethylhexyl alcohol, cyclohexane, benzene sulfonic acid and a polymerization inhibitor were contacted in a batch reactor 2 at a temperature range of about 85-120° C. As 2-ethylhexyl acrylate is produced; the temperature of the mixture changes, increasing the boiling point of the solution. Because of impurity formation, or byproduct formation (i.e., heavy ends impurities), the reaction goes to approximately 95% 2-ethylhexyl alcohol conversion. All unreacted acrylic acid is lost to waste processing or within the finishing system of the process. Unreacted 2-ethylhexyl alcohol typically remains within the system, ultimately building up in the light ends recovery tower, column 10, as shown in FIG. 1.

The unreacted alcohol had traditionally been either burned, processed as waste, or held in inventory tanks to be purified prior to reuse for the esterification reaction. Utilizing the purification method, approximately 50-80% of the unreacted alcohol was recovered and recycled to the reactor. When using one tower for light ends recovery and 2-ethylhexyl alcohol recovery, during high reaction rates for the production of 2-ethylhexyl acrylate it is difficult to maintain inventory for purification and reuse. During periods of high rates, the unreacted alcohol was primarily burned resulting in production efficiency losses for the operation. Maintaining unreacted alcohol in inventory tanks did not eliminate the problem of 2-ethylhexyl acetate impurity. The close boiling points of acetate and desired acrylate product make it difficult to separate these two components.

The reaction mixture stream 20 was directed to an extractor 4 for removal of the sulfonic acid catalyst and some of the unreacted acrylic acid. Extraction occurs with water, however a combination of sodium hydroxide/water may also be used. Bases that could be used in the extraction process include sodium hydroxide, potassium hydroxide and ammonia at a normality of about 0.1-3.0. The reaction mixture stream 20 directed to the extractor generally has a composition of about 60-80% 2-ethylhexyl acrylate product, 15-30% cyclohexane solvent, 1.5-4% 2-ethylhexyl alcohol, and 1-2% acrylic acid. The residue stream 22 from extractor 4 is primarily water and directed to waste treatment. While a stream 24 taken from the top portion of extractor 4 is directed to a cyclohexane stripper column 6.

Column 6 serves to remove, purify and prepare for reuse in the reaction, cyclohexane solvent. Other solvents, such as benzene, heptane, toluene, and the like, as well as utilizing 2-ethylhexyl alcohol as a reactant solvent, may be used as solvents in the reaction. It has been found that cyclohexane solvent has a desired azeotrope with water, allowing for easier removal of the solvent from stream 24. Recycling the cyclohexane from column 6 to the reactor improves the usage of cyclohexane and allows for temperature control in the reactor 2. The top temperature of column 6 ranges from about 65-75° C. The base temperature of column 6 ranges from about 98 to about 104° C. Temperature control is important in column 6 so as to avoid solvent in the desired acrylate product residue, stream 26, or product in the column overhead in stream 28. Residue stream 26 is directed to a cooler (not pictured) to allow for better decantation/separation of phases. The overhead stream 28 of column 6 contains primarily cyclohexane and water which is condensed, the water decanted, and solvent recycled to reactor 2.

Residue stream 26 is directed to a decanter, not pictured, allowing for a separation of the aqueous and organic phases of the product stream. This mixture is cooled to a temperature of about 20-50° C. The organic phase of Stream 26 contains predominantly 2-ethylhexyl acrylate (about 90% of the stream), 2-ethylhexyl alcohol (less than about 4%, typically about 1-4%), smaller amounts (about 2-4%) of light and heavy ends impurities (as defined herein), and 2-ethylhexyl acetate (about 0.5-2%). The aqueous phase is directed to waste processing treatment, while the desired organic phase containing the acrylate product and unreacted 2-ethylhexyl alcohol is directed to a light ends removal column 8.

Light end impurities are removed in column 8. Light end impurities include 2-ethylhexyl alcohol, 2-ethylhexyl acetate, HAc, and small concentrations of cyclo, heptanol, other light alcohols and water, and 2-ethylhexyl aldehyde. Also contained in the light end impurities are small amount (e.g., less than 1000 ppm) of various ethers. The overhead stream 48 generally consists of about 25% -45% 2-ethylhexyl acrylate, 35-55% 2-ethylhexyl alcohol, 10-20% 2-ethylhexyl acetate, 2-4% cyclohexane, 1-3% acrylic acid and about 1-10% water and is directed to a decanter or vessel 14 for separation of the aqueous and organic phases. The top of column 8 generally operates at a range of about 105-120° C., while the bottom portion of the tower operates at a temperate range of about 145-165° C. The column operates under a vacuum of about 35-55 mmHg absolute (35-55 Torr).

Prior to entering vessel 14, stream 48 is cooled to a temperature of about 20-50° C. The majority of the material in vessel 14 is the organic phase which is directed for further processing. The small amount of aqueous phase from vessel 14 is directed to waste treatment, while the majority organic or acrylate product stream is directed, as stream 30, to column 10, the light ends recovery tower.

The hot residue stream taken from the bottom of column 8 is directed as stream 38 (1-3% heavy ends, 500-1400 ppm 2-ethylhexyl acetate, the remainder is 2-ethylhexyl acrylate, and inhibitors) to column 12, the specification tower where heavy ends impurities are removed from the acrylate product. The top of column 12 operates at a temperature generally of about 135-150° C., while the base temperature of the column operates at about 155-175° C. Tower 12 top pressure ranges from 75-100 mmHg absolute. Heavy ends impurities consist primarily of 2-ethylhexyl-3-acryloxy propionate, 2-ethylhexyl-3-(2-ethylhexoxy)propionate, and polymerization inhibitors. The overhead, stream 40, of column 12 consists primarily of specification grade 2-EHAcA, 2-ethylhexyl acrylate product (99.5+% 2-EHAcA, 300-2000 ppm 2-EHAc, 100-500 ppm 2-EHOH, plus minor amounts of acrylic acid, inhibitors, and heavy ends impurities).

Generally, the reaction process contains polymerization inhibitors. For the present invention, at least one inhibitor is employed in the reactor for use during the reaction of components. At least one inhibitor is also added to the purification system of the process. The inhibitor may be added as appropriate in accordance with the equipment being utilized. The inhibitor (s) are added generally where a stream is heated or condensed. For the present invention, inhibitors were added in the acrylic acid feed to the reactor vessel 2, as well as to columns 8, 10, 12, and 16.

Examples of inhibitors include those such as hydroquinone, mono methyl ether of hydroquinone, and phenothiazine (PTZ). An alternate to the above-described inhibitors is air which can be injected into the base of the distillation tower to act as a vapor phase polymerization retarder. This air inhibitor may be used alone in combination with the above inhibitors during operation. Alternatively, air combined with an inert stream, such as nitrogen, may be provided as a blend gas. The oxygen concentration levels for the blend gas is typically about 5-10% by volume.

Additional inhibitors contemplated for use in the present invention are characterized by the presence of at least one other substituent on the benzene ring. Such other substituent serves to activate the phenolic inhibitor. Representative substituents include C₁₋₄ alkoxy such as methoxy and ethoxy. Other substituents include hydroxyl, sulfhydryl, amino, C₁₋₉ alkyl, phenyl, nitro, or N-linked amide, for example. Exemplary phenolic inhibitors include, but are not limited to, p-methoxyphenol (MEHQ), hydroquinone, or catechol such as tertiary butyl catechol or di-tertiary butyl catechol.

Concentrations of inhibitors are given herein in parts per million (ppm) by weight. Phenolic inhibitors are typically added to result in concentrations ranging from about 10 ppm to about 1500 ppm, however embodiments may include about 20 ppm to about 1000 ppm, as well as about 50 ppm to about 600 ppm, and about 150 to about 250 ppm in the inhibited mixture.

A coinhibitor may be employed along with the inhibitor, and is generally described as a metal cation having at least two valence states which are interconvertible via electron transfer reactions with other species (e.g., radicals) in the mixture. That is, the two valence states have similar enough thermodynamic stabilities to allow a cyclic, or reversible, electron transfer to occur. Representative examples of such metal cations include, but are not limited to, manganese, copper, chromium, cerium, iron, or combinations thereof. Preferably, the metal cation is a cation of manganese (Mn). The term manganese (Mn), as used herein, refers to the active species of metal cation.

The coinhibitor is added such that the concentration present in the inhibited mixture is about 0.1 ppm to about 100 ppm. Alternate embodiments of concentration for coinhibitor include a range of about 1 ppm to about 50 ppm, as well as a range of about 1 ppm to about 20 ppm, or, about 2 ppm to about 10 ppm.

Another class of polymerization inhibitors is the family of stable, radical scavengers such as TEMPO as provided by Nalco Exxon Corporation, or Ciba-Geigy Company.

The bottom residue stream 42 of column 12 consists primarily of about 2-ethylhexyl acrylate (about 50-80%); about 15-40% heavy ends impurities, and minor amounts of catalyst and inhibitors making up the balance of the stream. Stream 42 is directed to column 16, wherein heavy ends impurities are removed. The base temperature of the column operates at about 135-160° C. Column 16 operates at a pressure of about 10-25 mmHg absolute (10-25 Torr). The residue stream 46 of column 16 consists primarily of heavy ends impurities and about 1-20% 2-ethylhexyl acrylate and is directed to waste treatment. The overhead stream 44 of column 16 consists predominately of 2-ethylhexyl acrylate and is directed for recycle into the purification system. It is shown in FIG. 1 to be re-entered into the system by blending with stream 26 for entry into column 8, the light ends removal tower. However, those of skill in the art will recognize that stream 44 may be blended into the purification system at any appropriate point in accordance with the equipment utilized.

Once the phases have separated in decanter or vessel 14, the cooled organic phase, stream 30, is preheated to a temperature of about 80-120° C. and directed to column 10, the light ends recovery tower. A continuous cycle exists among the residue stream 32 of column 10, entering into the feed or top half of column 8, processing through column 8, exiting as overhead stream 48, entering vessel 14, and the organic phase entering column 10. Residue stream 32 consists of about 45-98% 2-ethylhexyl acrylate product, about 1-30% 2-ethylhexyl alcohol, and about 1-25% 2-ethylhexyl acetate. A liquid side stream 36 is taken from the top one third of column 10, which consists predominately of 2-ethylhexyl alcohol. Overhead stream 34 of column 10 consists of predominately about 15-70% (40% on average) unreacted 2-ethylhexyl alcohol, about 5-20% of 2-ethylhexyl acetate, less than about 5% light ends impurities, and less than about 5% of 2-ethylhexyl acrylate. Stream 34 is generally considered a waste stream and processed as such.

If side stream 36 was not withdrawn from column 10, the overhead stream 34 would consist primarily of about 65% (on average) unreacted alcohol, with impurities, instead of about 40% (on average) as is seen with the side stream. An exemplary composition of stream 34, in the absence of a side stream withdrawal, was found to be about 70% 2-ethylhexyl alcohol, about 10% 2-ethylhexyl acetate, about 3% 2-ethylhexyl acrylate, and the remainder being light ends impurities. A direct recycle (without a side stream draw) of the overhead stream 34 to the reactor resulted in emulsion problems affecting interface levels in the decanter or vessels and extractors within the reaction and purification process. It is believed that light ends impurities were causing the emulsion problems. Efforts to remove the light ends impurities before the stream reached the light ends recovery tower 10, resulted in production efficiency losses.

The inventors have found that 2-ethylhexyl alcohol, having about 65-85% purity, can be recovered by extracting a liquid side stream from the upper one third of column 10. Stream 36 consists predominately of 2-ethylhexyl alcohol, about 10-20% 2-ethylhexyl acetate, about 5-15% 2-ethylhexyl acrylate and the remainder consisting of minor amounts of HAcA and other light end impurities. These other light end impurities are believed to be the emulsion causing impurities and have been found present at less than about 2%, usually less than about 1%. It has been recognized by the inventors that 2-ethylhexyl alcohol is most concentrated at the top third of column 10 wherein a side stream may be extracted having sufficiently pure 2-ethylhexyl alcohol to allow for reuse within the reaction system, without having to purify the alcohol as previously known in the art. If one tries to extract from a point below the feed to the column, primarily acrylate product is obtained. The middle third of the column 10 generally contains a combination of acrylate product and unreacted alcohol. There remains some close boiling 2-ethylhexyl acetate material in the alcohol side stream 36. However, the acetate is not in sufficient amount to significantly upset the esterification reaction or purification system during reuse of the alcohol.

While the emulsion causing impurity is not known, it appears that the exact impurity causing the emulsion problems is not present, or is present in an insufficient quantity to cause emulsion problems, when 2-ethylhexyl alcohol is withdrawn as a liquid side stream, such as shown for stream 36. In situations when the liquid side stream 36 is at about 65% purity, it remains acceptable for use in recycle into the reaction system due to its absence of emulsion causing impurity(ies).

The benefits of the present invention result in direct savings from:

1. elimination of process steps, i.e., second purification of 2-EHOH;

2. avoiding tank inventory issues, e.g. Storing unpurified 2-EHOH;

3. avoiding emulsion problems in the reaction and purification process; and,

4. column 10 serves multi functional purposes, i.e., removal of light ends impurities from the reaction mixture, recovery of 2-EHOH as side stream (stream 36) for recycle and reuse to the reactor, and recovery of 2-ethylhexyl acrylate (streams 32 & 38).

What is claimed is:

1. A method of recovering 2-ethylhexyl alcohol comprising
a) reacting in a reactor, acrylic acid with 2-ethylhexyl alcohol in the presence of a solvent, an acidic catalyst, and at least one polymerization inhibitor, to form a reaction mixture of primarily 2-ethylhexyl acrylate, acrylic acid, solvent, catalyst, 2-ethylhexyl alcohol and 2-ethylhexyl acetate;
b) directing the reaction mixture to an extraction process to remove at least a portion of the catalyst and at least a portion of the unreacted acrylic acid;
c) directing the mixture of step (b) to a process for removal of solvent and forming a stream comprising 2-ethylhexyl acrylate, 2-ethylhexyl alcohol, 2-ethylhexyl acetate, light ends impurities, and heavy ends impurities;
d) directing the stream of step (c) for removal of light ends impurities forming an overhead stream comprising 2-ethylhexyl acrylate and 2-ethylhexyl alcohol, and a residue stream comprising 2-ethylhexyl acrylate, heavy ends impurities, and 2-ethylhexyl acetate;
e) directing the overhead stream of step (d) to a light ends recovery column wherein the residue is recycled to the light ends removal process of step (d);
f) withdrawing a liquid side stream from the top third of the light ends recovery column to obtain a predominate 2-ethylhexyl alcohol stream.

2. The method of claim 1 wherein the 2-ethylhexyl alcohol of step (f) is directed to the reactor.

3. The method of claim 1 wherein the light ends impurities comprise 2-ethylhexyl alcohol, 2-ethylhexyl acetate, acetic acid, solvent, heptanol, water, 2-ethylhexyl aldehyde, acrylic acid.

4. The method of claim 1 wherein the heavy ends impurities comprise 2-ethylhexyl-3-acryloxy propionate, 2-ethylhexyl-3-(2-ethylhexoxy)propionate, and polymerization inhibitor(s).

5. The method of claim 1 wherein the 2-ethylhexyl alcohol is recovered from step (f) at a concentration level of about or greater than 65%.

6. The method of claim 5 wherein the 2-ethylhexyl alcohol is recovered from step (f) at a concentration level of greater than 75%.

7. The method of claim 6 wherein the 2-ethylhexyl alcohol is recovered from step (f) at a concentration level of greater than 85%.

8. The method of claim 1 wherein the solvent is selected from the group of cyclohexane, benzene, heptane, toluene, 2-ethylhexyl alcohol, phosphoric acid, and polyphosphoric acid.

9. The method of claim 1 wherein the catalyst is selected from the group of benzene sulfonic acid, methane sulfonic acid, sulfonic acid resin, p-toluene sulfonic acid, sulfuric acid, and phosphoric acid.

10. The method of claim 1 wherein at least one polymerization inhibitor is added to the reaction system and to the purification system, said inhibitor selected from the group air, air blend gas containing at least 5% oxygen by volume, hydroquinone, mono methyl ether of hydroquinone, phenothiazine (PTZ), methoxy benzene, ethoxybenzene, phenol, hydroxylbenzene, sulfhydrylbenzene, aminobenzene, C₁₋₉ alkylbenzene, p-methoxyphenol (MEHQ), tertiary butyl catechol and di-tertiary butyl catechol, a metal cation of manganese, copper, chromium, cerium, iron, TEMPO, and combinations thereof.

11. The method of claim 1 wherein the extraction solvent of step (b) comprises water.

12. The method of claim 11 wherein the extraction solvent comprises water and at least one of sodium hydroxide, potassium hydroxide and ammonia at a normality of about 0.1-3.0.

13. A method of recovering 2-ethylhexyl alcohol from a mixture comprising 2-ethylhexyl acrylate, 2-ethylhexyl alcohol, 2-ethylhexyl acetate, said method comprising feeding the mixture to a tower so as to withdraw as a liquid side stream, from the top third of the tower, a stream comprising at least 65% 2-ethylhexyl alcohol, about 10-20% 2-ethylhexyl acetate, about 5-15% 2-ethylhexyl acrylate, and having light ends impurities other than acrylic acid at a concentration of less than about 2%.

14. A process for the production of 2-ethylhexyl acrylate which includes a method of recycling unreacted 2-ethylhexyl alcohol comprising:
a) reacting in a reactor, acrylic acid with 2-ethylhexyl alcohol in the presence of a solvent, an acidic catalyst, and at least one polymerization inhibitor, to form a reaction mixture of primarily 2-ethylhexyl acrylate, acrylic acid, solvent, catalyst, 2-ethylhexyl alcohol and 2-ethylhexyl acetate;
b) directing the reaction mixture to an extraction process to remove at least a portion of the catalyst and at least a portion of the unreacted acrylic acid;
c) directing the mixture of step (b) to a process for removal of solvent and forming a stream comprising 2-ethylhexyl acrylate, 2-ethylhexyl alcohol, 2-ethylhexyl acetate, light ends impurities, and heavy ends impurities;
d) directing the stream of step (c) for removal of light ends impurities forming an overhead stream comprising 2-ethylhexyl acrylate and 2-ethylhexyl alcohol, and a residue stream comprising 2-ethylhexyl acrylate, heavy ends impurities, and 2-ethylhexyl acetate;
e) directing the overhead stream of step (d) to a light ends recovery column wherein the residue is recycled to the light ends removal process of step (d);
f) withdrawing a liquid side stream from the top third of the light ends recovery column to obtain a predominate 2-ethylhexyl alcohol stream which is recycled to the reactor.
